(19) United States
(12) Patent Application Publication
(10) Pub. No.: US 2020/0194106 A1
(43) Pub. Date: Jun. 18, 2020

(54) SYSTEM AND DEVICE FOR NON-INVASIVE DETECTION OF INPUT AND OUTPUT EVENTS

(71) Applicant: PERFORMANCE ATHLYTICS, Austin, TX (US)

(72) Inventors: Byron P. OLSON, Boone, IA (US); Nithin O. RAJAN, Austin, TX (US); Dustin M. FRECKLETON, Austin, TX (US); David E. CLIFT-REAVES, Austin, TX (US); James BREAUX, Austin, TX (US); Lane MUSGRAVE, Austin, TX (US); Paulo E. XAVIER DA SILVEIRA, Boulder, CO (US)

(73) Assignee: LVL TECHNOLOGIES, INC., Austin, TX (US)

(21) Appl. No.: 16/639,976

(22) PCT Filed: Aug. 16, 2018
(86) PCT No.: PCT/IB2018/056164

§ 371 Date: Feb. 18, 2020

Related U.S. Application Data
(60) Provisional application No. 62/546,466, filed on Aug. 16, 2017.

(51) Int. Cl. G16H 20/30; G16H 40/67; G16H 20/60; H04B 1/3827; H04L 29/08; A61B 5/00; A61B 5/0205

(52) U.S. Cl. CPC G16H 20/30; G16H 40/67; G16H 20/60; G06F 1/163; H04L 67/12; A61B 5/7285; A61B 5/02055; H04B 1/385

(57) ABSTRACT

Wearable device has at least one sensor operable to detect motion of the wearable device and a biological sensor coupled to the processor and operable to detect a biological indicator of the user. Wearable is operable to obtain at least one biological indicator of the user and correlate the biological indicator of the user with the detected motion in time. Wearable is also able to determine one or more input events based on one or more of the at least one biological indicator or detected motion, each input event including an input activity and input duration and create an input log for every determined one or more inputs events, wherein the input log includes an entry for each corresponding input event that includes at least one of the input activity, input duration, and input time. In at least one example, the wearable determines a net balance of the user.

### CROSS-REFERENCE TO RELATED APPLICATION

This application claims priority to U.S. Provisional Patent Application No. 62/546,466, which was filed in the U.S. Patent and Trademark Office on Aug. 16, 2017, all of which is incorporated herein by reference in its entirety for all purposes.

### FIELD

The present disclosure relates to systems and devices related to the non-invasive detection of biological input and output events.

### BACKGROUND

Wearable devices have been used by performance athletes and amateurs to monitor physical activities. Wearable devices can be configured to be coupled to a mobile device or external computer. The wearable device can include a wireless connection to the mobile device. The wearable device can include a sensor that is configured to measure motion of the user.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the present disclosure. It should be understood, however, that the present inventive concept is not limited to the precise examples and features shown. The accompanying drawings, which are incorporated in and constitute a part of this specification, illustrate an implementation of apparatuses consistent with the present inventive concept and, together with the description, serve to explain advantages and principles consistent with the present inventive concept.

FIG. 1A illustrates an example of a mobile device according to the present disclosure.

FIG. 1B illustrates an example of a wearable device according to the present disclosure.

FIG. 1C illustrates an example of a remote computer according to the present disclosure.

FIG. 1D is a schematic diagram of an example wearable device system according to the present disclosure.

FIG. 2 is a schematic diagram of an example mobile device system according to the present disclosure.

FIG. 3 is a schematic diagram of a mobile device and a display according to the present disclosure.

FIG. 4 is a flowchart presented in accordance with an example system.

FIG. 5 is a flowchart presented in accordance with another example system.

FIGS. 6A-C illustrate accelerometer data used to detect matter input from the x, y, and z axis of the accelerometer, respectively.

FIGS. 7A-C illustrate accelerometer data detecting actions other than input from the x, y, and z axis of the accelerometer, respectively.

FIGS. 8A-C illustrate accelerometer data detecting actions which affect heart rate from the x, y, and z axis of the accelerometer, respectively.

FIGS. 9A-C illustrate accelerometer data detecting a coughing action from the x, y, and z axis of the accelerometer, respectively.

FIG. 10 shows an example using heart rate to detect matter input.

FIG. 11 shows a second example using heart rate to detect matter input.

FIG. 12 shows a third example using heart rate to detect matter input.

FIG. 13 shows a fourth example using heart rate to detect matter input.

### DETAILED DESCRIPTION

Several definitions that apply throughout this disclosure will now be presented. The term “comprising” means “including, but not necessarily limited to”; it specifically indicates open-ended inclusion or membership in a so-described combination, group, series and the like. “About” refers to almost, nearly, on the verge of, or without significant deviation from the numeric representation. For example, about 20 can be 20, or a small deviation from 20. “Coupled” refers to the linking or connection of two objects. The coupling can be direct or indirect. An indirect coupling includes connecting two objects through one or more intermediary objects. Coupling can also refer to electrical or mechanical connections. Coupling can also include magnetic linking without physical contact.

The present disclosure endeavors to solve a variety of problems in the industry. The present disclosure includes the ability to monitor input events and output events. The present disclosure also allows the monitoring of the net input and output balance of an individual over a given period of time. In at least one example the present disclosure provides for long-term monitoring of the balance, which is useful in monitoring health, well-being, and aids in achieving health-related goals for a user.

The present disclosure includes a system and device for determining input events and output events, such as matter ingestion and excretion using non-invasive techniques. Input events can include one or more of eating, drinking, smoking, or inhaling a mist or gas, temperature gain, muscle gain, bone mass gain, fat gain, calories gained, sleep gain, attention gain, alertness gain, laughing—an indicator of mood improvement, or the like. Output events can include one or more of defecation, urination, sweating, evaporation (including evaporation through breathing), vomiting, diarrhea, sneezing, coughing, yelling or crying—indicators of mood deterioration, or the like.

The present disclosure can be implemented in one or more of the devices and/or systems described herein. In one example, the present disclosure includes a wearable device. As used herein, a wearable device is any device that is in contact or close proximity to a user of the device. Examples of wearable devices include a wrist worn device, a chest strap, clothing, an athletic aid, a monitor, a bracelet, a ring, compression sleeves, glasses or a head-mounted display, a headphone or an earphone. The wearable devices can be configured to have a wireless communication or wired communication interface to allow for exchange of data. In at least one example, the wearable device is operable to be electronically coupled to a mobile device. In at least one example, the wearable device can be configured to include a user notification component that provides instructions to the user. The user notification component can be a display, an audio device, a vibration device, or a visual indicator. In other examples, the user notification component can be omitted and the wearable device can communicate instructions to the mobile device for communication of the instructions to the user.

The term mobile device can include a device that has a processor and a memory. The mobile device in at least some examples includes a display. Additionally, the mobile device can include a communication component that is operable to allow for communication with the mobile device to an external device. The wearable device can also be configured to communicate with one or more external sensor components. The wireless communication can be performed using short range wireless communication protocols such as BLUETOOTH, ZIGBEE, Advanced and Adaptive Network Technology (ANT+), WI-FI, Radio Frequency Identification (RFID), or the like.

In an example, a mobile device system includes a mobile device and a wearable device and is operable to provide recommendations on input for a user. The mobile device has at least one sensor which can detect motion of the mobile device. The wearable device can detect a biological indicator of the user and can transmit the data to the mobile device. The mobile device, or another component in the system, correlates the biological indicator of the user with the detected motion in time to determine if one or more input event or one or more output event has occurred and creates an input and an output log, respectively, for each event. In at least one example, the mobile device also determines a net balance of the user based on the input and output logs. The net balance can provide the benefit of helping to improve the health and well-being of a user by being within a predetermined range, or below or above a predetermined threshold. For example, the net balance can be used to help a user to reach health-related goals such as, for example, staying well hydrated or helping the user to lose weight. To be well hydrated, a user should be above a hydration threshold. To lose weight, a user may need to remain below a total caloric threshold (for example, caloric intake minus caloric expenditure) or within a total caloric range. Although the system and device are described with respect to a mobile device, the system and device can be entirely operable on a wearable device.

In another example, a wearable device operable to provide recommendations to a user includes at least one sensor operable to detect motion of the wearable device. The wearable device can further include a processor coupled to the at least one sensor and a biological sensor coupled to the processor and is operable to detect a biological indicator of the user. The wearable device can also include a memory that is operable to store instructions to cause the wearable device to do one or more of the following: obtain at least one biological indicator of the user, correlate the biological indicator of the user with the detected motion in time, determine one or more input or output events based on one or more of the at least one biological indicator and/or detected motion, each event including an activity and a duration, or determine a net balance of the user based on the determined one or more events.

In another example, a mobile device can be operable to do one or more of the following: determine habits of a user and/or make recommendations to a user. The mobile device can include one or more internal sensors operable to detect at least one of motion of the mobile device or location of the device. The mobile device can further include a processor coupled to the one or more internal sensors and a biological sensor coupled to the processor and operable to detect a biological indicator of the user. The mobile device can also include a display coupled to the processor and operable to display data received from the processor. The mobile device can also include a memory coupled to the processor and operable to store instructions to cause the processor to do one or more of the following: obtain at least one biological indicator of the user, correlate the at least one biological indicator of the user with the detected motion in time, determine one or more input and/or output events based on one or more of the at least one biological indicator or detected motion, each event including an activity and a duration, or determine a net balance of the user based on the determined one or more events.

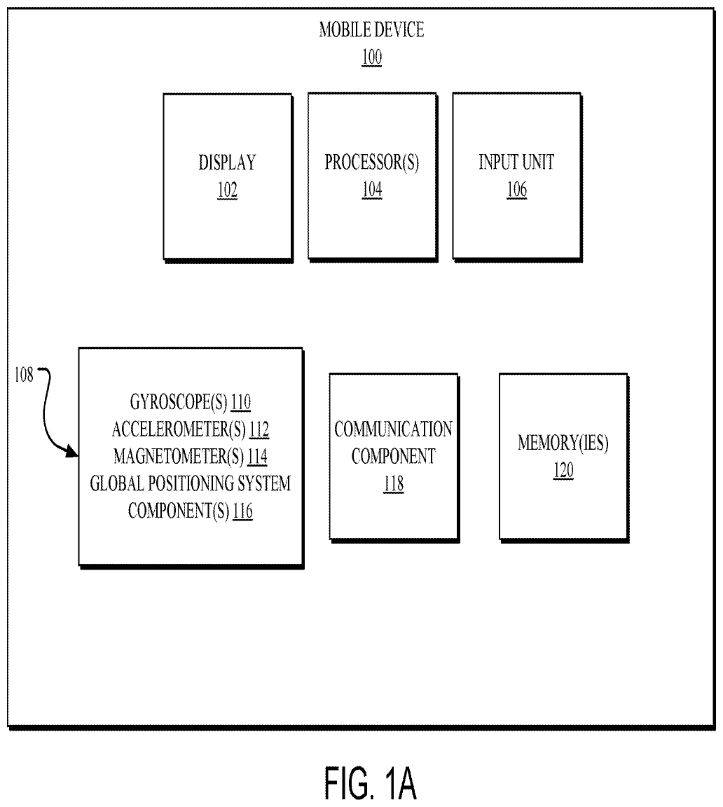

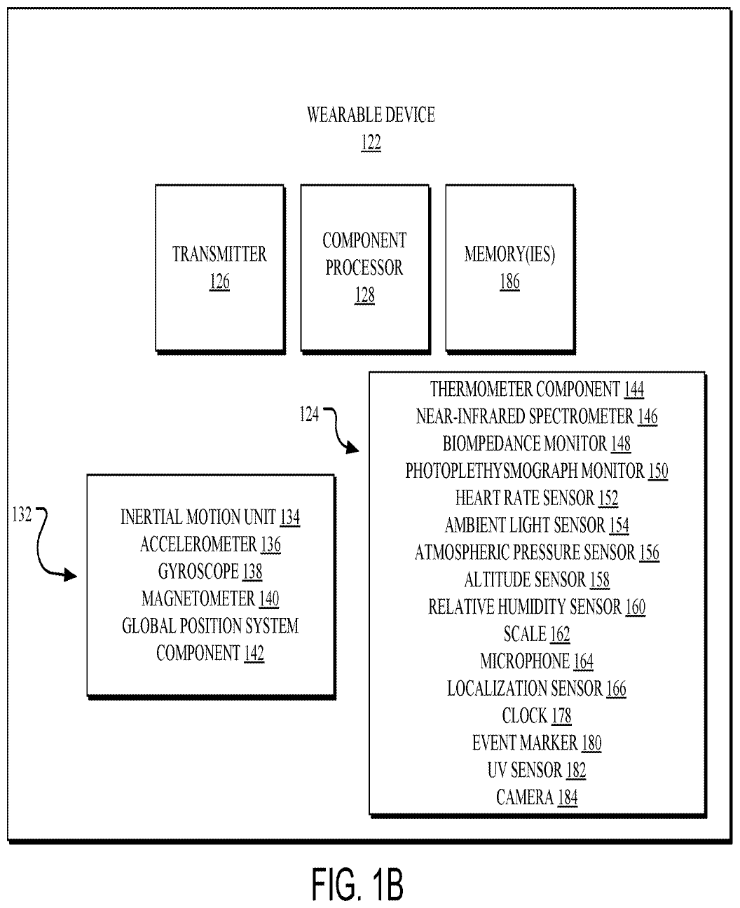

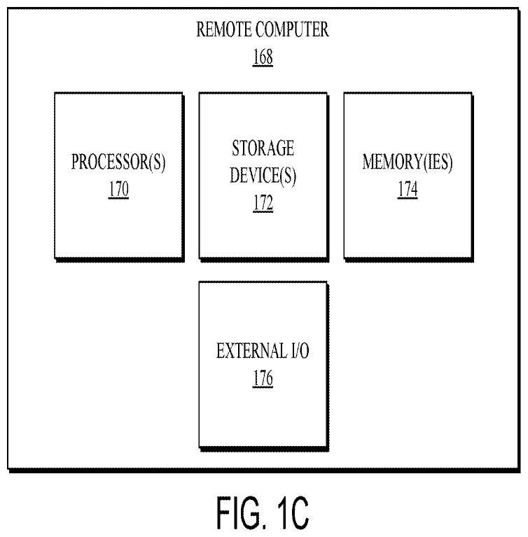

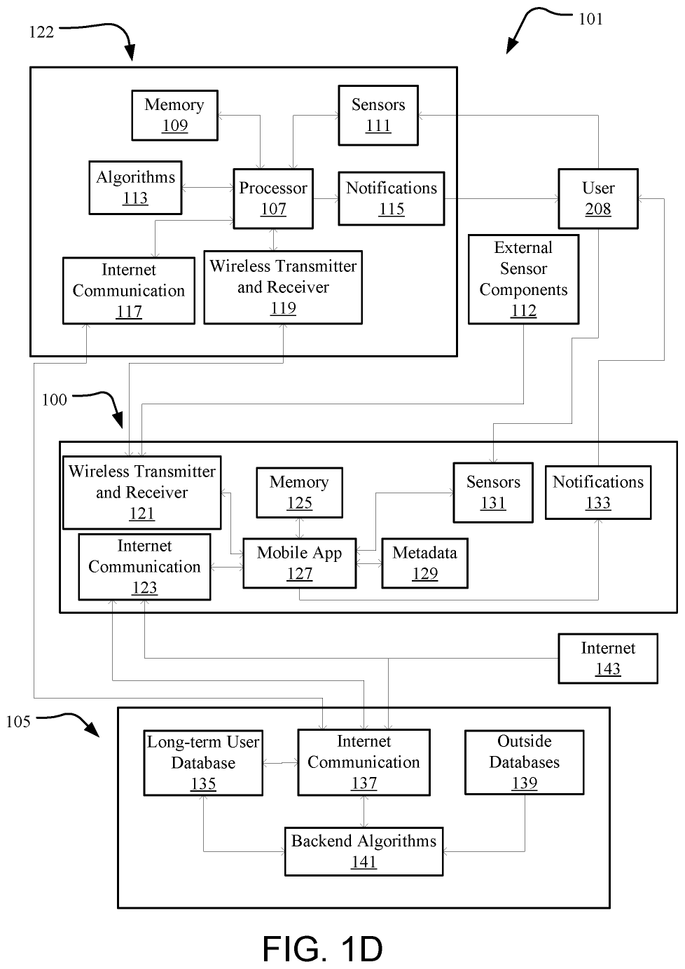

FIG. 1A illustrates an example of a mobile device 100 according to the present disclosure. The mobile device 100 includes a display 102, a processor 104, an input unit 106, at least one sensor 108, at least one communication component 118, and a memory 120. The at least one sensor 108 is operable to detect motion of the mobile device 100. The at least one sensor 108 can be a gyroscope 110, an accelerometer 112, a magnetometer 114, and/or a global positioning system component 116. The at least one communication component 118 is operable to receive data from a wearable device 122 or a remote computer 168. The processor 104 is coupled to the at least one sensor 108 and the at least one communication component 118.

FIG. 1B illustrates an example of a wearable device 122 according to the present disclosure. The wearable device 122 can include a transmitter 126, a component processor 128, a biological sensor 124, a memory 186, and additional sensors 132. The wearable device 122 can include at least one external sensor component which can be one or more of: scales, water bottles, glucose measurement systems, blood pressure monitors, pulse oximeters, respiration rate monitors, tissue oximeters, respirators, electrocardiogram monitors, or the like. The wearable device 122 can be enabled to wirelessly communicate with other devices. The biological sensor 124 can be coupled to the component processor 128 and is operable to detect a biological indicator 206 of a user 208. The transmitter 126 is operable to transmit a detected biological indicator 206 to the at least one communication component 118 of the mobile device 100, a remote computer 168, and/or another external device. The biological sensor 124 can be one or more of a thermometer component 144 operable to measure a temperature of skin of the user 208 and/or surrounding ambient temperature, a near-infrared spectrometer (NIRS) 146 operable to monitor chromophores that constitute a tissue of the user 208, a bioimpedance monitor 148, a photoplethysmograph (PPG) monitor 150, a heart rate monitor 152, an ambient light sensor 154, an atmospheric pressure sensor 156, an altitude sensor 158, a relative humidity sensor 160, a scale 162, a microphone 164, a localization sensor 166, a clock 178, an event marker 180, a ultra violet (UV) sensor 182, and/or camera 184. Furthermore, the biological sensor is operable to detect one or more of a heart rate, a heart rate variation, a respiration rate, a blood oxygen saturation level, muscle oxygenation level, skin temperature, skin perfusion, skin impedance, galvanic skin response, blood pressure, tissue perfusion, blood flow, blood volume, extracellular fluid, intracellular fluid, photoplethysmograph, calorie expenditure, activity detection, water loss, sweat rate, drink detection, drink volume detection, eating detection, eating volume detection, images, videos and/or sounds associated with a biological input or output event. The additional sensors 132 can be one or more of: an inertial motion unit (IMU) 134 including at least one of an accelerometer 136, gyroscope 138, magnetometer 140, and/or a global position system component 142.

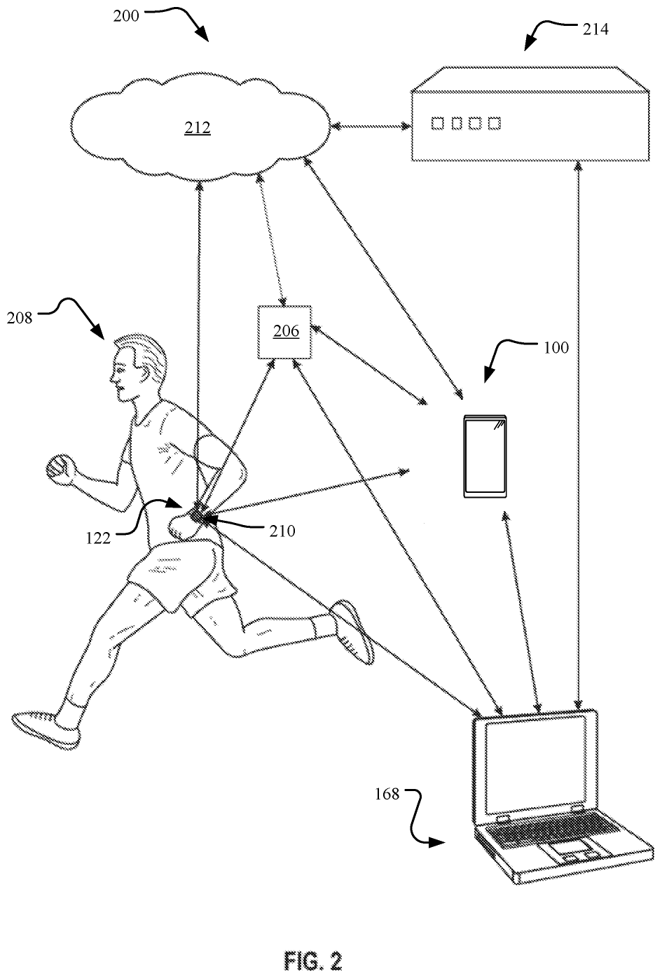

FIG. 1C illustrates an example of a remote computer 168. The remote computer 168 can include one or more of: one or more processors 170, one or more storage devices 172, one or more memories 174, or one or more external Input/Output (IO) interfaces 176. The remote computer 168 can be a cloud based computer system 212, shown in FIG. 2 or a cloud storage and data processing system 105, shown in FIG. 1D.

FIG. 1D is a schematic diagram of an example wearable device system 101 according to the present disclosure. The wearable device system 101 can include the mobile device 100, the wearable device 122, and/or a cloud storage and data processing system 105. In at least one example, the cloud storage and data processing system 105 can include one or more of the components described in relation to the remote computer 168 of FIG. 1C. Further, an internet 143 is operable to allow communication between the mobile device 100, the wearable device 122, and/or the cloud storage and data processing system 105. The wearable device 122 can include one or more of: a processor 107 operable to communicate with a memory 109, one or more sensors 111, one or more algorithms 113, internet communication 117, and/or a wireless transmitter and receiver 119. In one example, the one or more sensors 111 collects data from a user 208 and the processor 107 processes the data and sends at least one notification 115 to the user 208. The at least one notification 115 can be provided to the user 208 via one or more of a display, lights, sound, vibrations, and/or buzzers. The at least one notifications 115 can further be associated with achieving one or more predefined goals, wherein the one or more predefined goals are health or well-being. In one example, the predefined goal can be to improve well-being by exercising daily, such as walking 2-3 miles a day, in order to increase a user's overall health. In another example, the predefined goal can be more specific based on input and output events and suggest that a user to eat a specific quantity of a specific food at a specific time of the day, which can aid in increasing a user's overall health and well-being. In another example, the predefined goal can be to stay hydrated within an allowable range of net hydration balance, thus preventing disease states related to dehydration. In other examples, the predefined goal can include one or more goals, which can be both diet and exercise related.

The mobile device 100 includes a mobile application 127 operable to communicate with one or more of a memory 125, a wireless transmitter and receiver 121, a metadata 129, a one or more sensors 131, and an internet communication 123. In an example, the mobile device 100 is controlled by the mobile application 127 that collects additional data from the one or more sensors 131 and also collects the metadata 129. The metadata 129 can be, for example, from one or more of a user's calendar, contacts, or geographic location. For example, the mobile application 127 can access specific events on the user calendar to aid in the determination of whether or not the user is undergoing an input or output event at a given time. For example, references to words such as “Lunch”, “Dinner”, “Breakfast” are associated with a higher probability of eating and/or drinking while words such as “Run”, “Workout”, “Spin class” are more closely associated with output events that lead to a loss of hydration volume due to increased physical activity, resulting in a higher loss of liquids due to increased perspiration and respiration rate. The mobile application 127 may also use one or more of the user contacts and calendar to determine whether the user is in the presence of one or more people with whom the user experiences input or output events. Moreover, the mobile application 127 may use the user's geographic location to assist in the estimate of whether the user is likely to undergo an input or output event. For example, the probability of the user ingesting food or drinking is higher when he/she is in a restaurant, bar, cafe, or cafeteria. The mobile application 127 can also send one or more notifications 133 to the user 208. The notifications 133 can also be provided to the user 208 via one or more of a display, lights, sound, vibrations, or buzzers.

The cloud storage and data processing system 105 can include one or more backend algorithms 141 operable to communicate with a long-term user database 135, one or more outside databases 139, or an internet communication 137. The cloud storage and data processing system 105 enables the storage of long-term user data into the long-term user database 135 and the execution of more complex backend algorithms 141. These backend algorithms 141 also benefit from the long-term data derived from other users that are similar to a specific user. The information derived from the backend algorithms 141 are provided to the user 208 either via the mobile application 127 or directly to the wearable device 122.

FIG. 2 illustrates an example mobile device system 200. The mobile device system 200 can include a mobile device 100, one or more wearable devices 122, a remote computer 168, a cloud-based computer system 212, and/or a storage device 214. The components can communicate with each other as indicated by the arrows shown. For example, the mobile device 100 can communicate with one or more of the cloud based computer system 212, the remote computer 168, or the one or more wearable devices 122.

In one example according to the present disclosure, the one or more wearable devices 122 can be in the form of a wrist device 210 operable to be worn on a wrist of a user 208. The wrist device 210 can also include additional sensors 132 (shown in FIG. 1B) to measure motion of a wrist. The detected motions then can be transmitted to the mobile device 100 or remote computer 168. Example studies concerning motions of the wrist will be discussed further below. The wrist device 210 can also be operable to communicate with the mobile device 100 or other connected device via a wired or wireless communication connection. For example, the wrist device 210 can wirelessly communicate with the mobile device 100, the remote computer 168, or a cloud based computer system 212 indicated by the arrows shown in FIG. 2. In another example, the wrist device 210 can communicate with the mobile device 100, the remote computer 168, or the cloud based computer system 212 via a wired connection. The wrist device 210 can be entirely self-sufficient. In other examples, the wrist device 210 can be without a connection to the internet and/or mobile device 100. The data transmitted to the cloud based computer system 212 or other long-term memory storage device can be stored for future use and/or processed to provide information useful to a user 208.

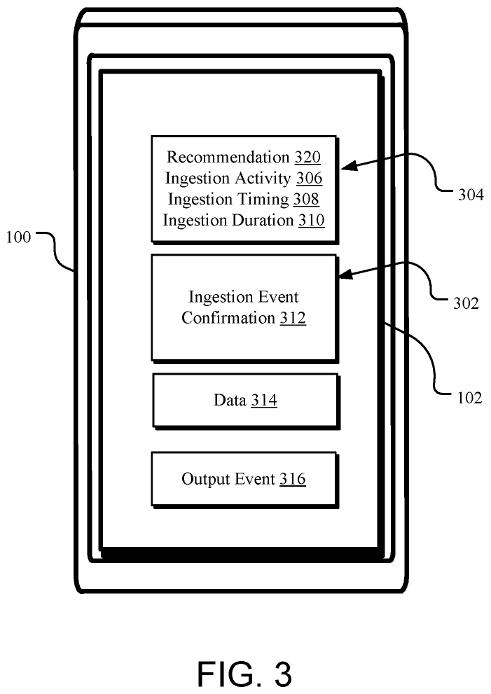

FIG. 3 is a schematic diagram of a mobile device 100 and display 102 according to the present disclosure. Although shown on a mobile device 100, the display 102 can be a display 102 of any device such as, for example, the wearable device 122. The memory 120 of the mobile device 100 can be operable to store further instructions to cause the mobile device 100 to display a recommendation 320 to the user 208 for a next input event that includes an input activity 306, an input timing 308, and an input duration 310. For example, the mobile device 100 or wearable device 122 can display instructions to drink two ounces of water in about five minutes while a user 208 is running. The input activity 306 can be an intake of a solid, liquid, or gas. Then input activity 306 can further be at least one of: eating, drinking, smoking, or inhaling a mist and/or gas, temperature gain, muscle gain, bone mass gain, fat gain, calories gained, sleep gain, attention gain, alertness gain, laughing—an indicator of mood improvement, or the like. Furthermore, the memory 120 of the mobile device 100 can cause the mobile device 100 to display a determined input event 302 on a display of the mobile device and receive confirmation 312 or modification of the displayed input event 302. Also, the display 102 can display data 314 received from the remote computer 168, the cloud based computer system 212, or the one or more wearable devices 122.

The display 102 can also display a recommendation 320 to the user for a next output event 316 that includes one or more of: an output activity, an output timing, and/or an output duration. For example, the display 102 can display instructions to perform a high cardio activity for ten minutes at a certain time in the morning to induce calorie loss. The output events 316 can be perspiration, urination, defecation, excretion, coughing, sneezing, vomiting, blood loss, plasma loss, ascetic fluid loss, fluid redistribution, diarrhea, temperature loss, temperature change, insensible fluid loss, fat loss, muscle loss, bone loss, calories burnt, sleep loss, attention loss, alertness loss, or yelling or crying (indicators of mood loss or the like).

Furthermore, the net balance of input and/or output can be displayed. The long-term monitoring of the net balance of input events 302 and output events 316 can be used by the mobile device 100 and/or wearable device 122 to provide the user 208 with relevant information regarding their health and wellness. The beneficial information includes at least one of: hydration balance, weight loss, muscle mass management, weight management, sleep deficit management, attention deficit management, calories accumulated, bone mass levels, smoke cessation, and/or temperature management. For example, the mobile device 100 and/or the wearable device 122 can use sensed estimates of net food balance and current estimates of user input activity (in this example, eating) to notify the user to stop eating during the course of a meal in order to help the user achieve a predetermined weight loss target.

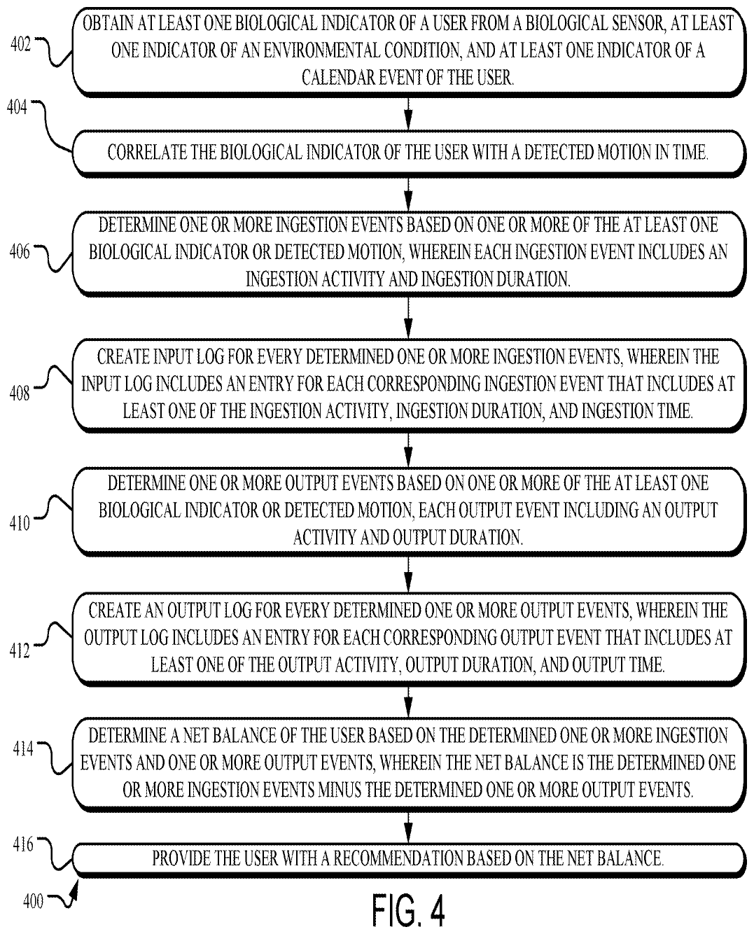

Referring to FIG. 4, a flowchart is presented in accordance with an example system. The example method 400 is provided by way of example, as there are a variety of ways to carry out the method 400. The method 400 described below can be carried out using the configurations illustrated in FIGS. 1-3, for example, and various elements of these figures are references in example method 400. Each block shown in FIG. 4 represents one or more processes, methods or subroutines, carried out in the example method 400. Furthermore, the illustrated order of blocks is illustrative only and the order of the blocks can change according to the present disclosure. Additional blocks may be added or fewer blocks may be utilized, without departing from this disclosure. The example method 400 can begin at block 402.

Block 402 obtains at least one biological indicator 206 of a user 208 from a biological sensor 124 (for example, from wearable device 122), at least one indicator of an environmental condition, and at least one indicator of a calendar event of the user. Block 404 correlates the biological indicator 206 with a detected motion in time. Block 406 determines if one or more input events 304 occurred based on the biological indicator 206 or detected motion. Each input event 304 includes at least one of: an input activity 306 or input duration 310. Block 408 creates an input log for every determined one or more input events 304, wherein the input log includes an entry for each corresponding input event that includes at least one of: the input activity 306, input duration 310, or input time 308. Block 410 determines if one or more output events 316 occurred based on one or more of the at least one biological indicator 206 or detected motion, wherein each output event 316 including at least one of: an output activity or output duration. Block 412 creates an output log for every determined one or more output events 316, wherein the output log includes an entry for each corresponding output event that includes at least one of the output activity, output duration, or output time. Block 414 determines a net balance of the user 208 based on the determined one or more input events and/or one or more output events, wherein the net balance is the determined one or more input events minus the determined one or more output events. In at least one or more examples, no input events can be present. In other examples, no output events can be present. Block 416 provides the user 208 with a recommendation 320 based on the net balance. The recommendation 320 can be, for example, “drink 16 oz of water”, “go to bed 1 hour earlier this evening”, “eat more vegetables and fruits during dinner”, or the like. The net balance can also be used to provide pertinent information to a user 208 who can be participating in a weight loss program, hydration management program, sleep management program, mood management program, or the like. For example, monitoring calories and mass net intake from day-to-day is part of a weight loss program. In another example, monitoring hydration intake and output for long-distance runners is important to maintain a certain level of hydration.

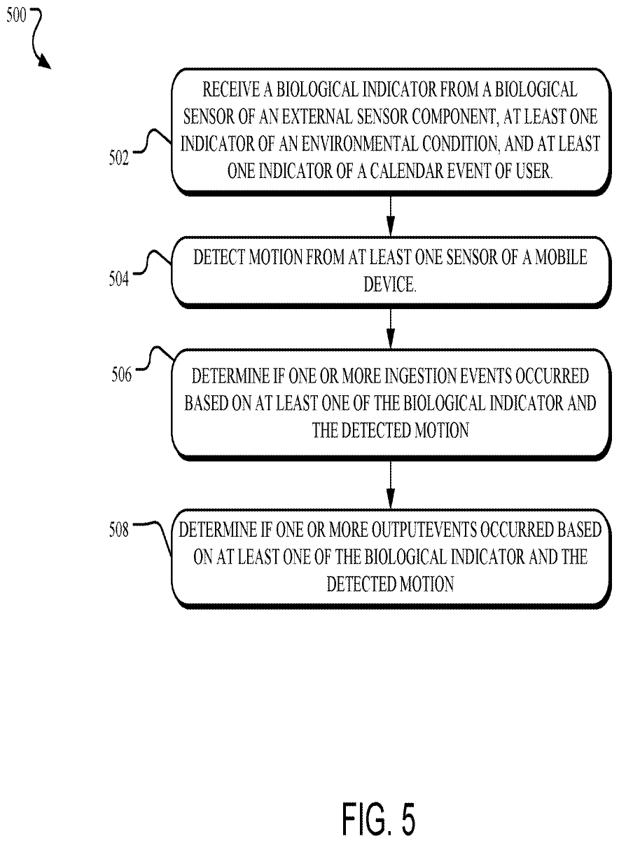

Referring to FIG. 5, another flowchart is presented in accordance with an example system. The example method 500 is provided by way of example, as there are a variety of ways to carry out the method 500. The method 400 described below can be carried out using the configurations illustrated in FIGS. 1-3, for example, and various elements of these figures are references in example method 500. Each block shown in FIG. 5 represents one or more processes, methods or subroutines, carried out in the example method 500. Furthermore, the illustrated order of blocks is illustrative only and the order of the blocks can change according to the present disclosure. Additional blocks may be added or fewer blocks may be utilized, without departing from this disclosure. The example method 500 can begin at block 502.

Block 502 receives a biological indicator 206 from one or more of: a biological sensor 124 of an external sensor component 122, at least one indicator of an environmental condition, and/or at least one indicator of a calendar event of a user. Block 504 detects motion from at least one sensor 108 of the mobile device 100. In at least one example, motion can be detected from one or more sensors 132 of the wearable device 122. Block 506 determines if one or more input events 304 occurred based on the at least one of the biological indicator 206 and the detected motion. Block 508 determines if one or more output events 316 occurred based on the at least one of the biological indicator 206 and detected motion. While the method illustrated includes detecting both input events and output events, other examples can include just one of input events or output events.

The use of at least one sensor 108 or biological sensors 124 can be used in isolation or in combination. For example, the mobile device 100 can obtain data from the IMU 134, wherein the determination of one or more input events is based on the obtained data from the IMU 134 with respect to time and/or heart rate. In at least one example, the time of a heart rate spike and motion detected from the IMU 134 can indicate the start of an input event 304. Furthermore, predetermined motions, such as a return to a position prior to initiation of the input event 304, for example, can indicate an end of an input event 304.

In at least one example according to the present disclosure, a mobile device 100 is operable to determine habits of a user 208 and make recommendations 320 regarding changes in habits. The mobile device 100 includes one or more internal sensors 108 operable to detect at least one of a motion of the mobile device 100 or location of the device. The mobile device 100 also includes a processor 104 coupled to the one or more internal sensors 108 and a display 102 coupled to the processor 104 and operable to display data 314 received from the processor 104. The communication component 118 is coupled to the processor 104 and is operable to receive data 314 from at least one of: a remote computer 168 or one or more external sensor components 122 operable to detect a biological indicator 206. The mobile device 100 further includes a memory 120 coupled to the processor 104 and is operable to store instructions to cause the processor to perform the process of logging input events method 400 according to FIG. 4. In at least one example, the wearable device 122 is operable to determine habits of a user 208 and make recommendations 320 regarding changes in habits without the use of a mobile device 100 and/or a remote computer 168.

In at least one example according to the present disclosure, a mobile device system 200 is operable to provide recommendations 320 on input for a user 208 including one or more of: a mobile device 100 and an external sensor 122. The mobile device 100 includes at least one sensor 108 operable to detect motion of the mobile device 100 and at least one communication component 118 operable to receive data 314 from one or more external sensor components 122 or remote computer 168. The mobile device 100 also includes a processor 104 coupled to the at least one sensor 108 and the at least one communication component 118. The mobile device system 200 also can include one or more of: an external sensor component 122 having a component processor 128; a biological sensor 124 coupled to the component processor 128 and operable to detect a biological indicator 206 of the user 208; or a transmitter 126 operable to transmit the detected biological indicator 206 to the at least one communication component 118 of the mobile device 100. The remote computer 168 includes a processor 170 and a memory 174 that is operable to store instructions to perform the process of logging input events 400 according to FIG. 4.

### Experimental Results

To test the ability of using one or more wearable devices or external sensor components 122, and also the ability of using at least one biological indicator or detected motion, 406 and 506, respectively, to detect matter input, matter input trials were performed using an accelerometer and a heart rate monitor.

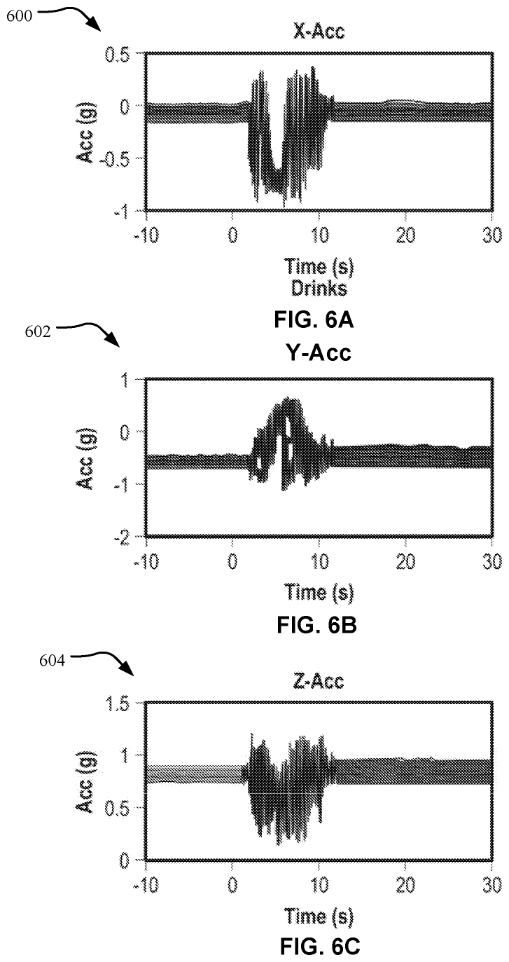

In an example study using an accelerometer, FIGS. 6A-C illustrate accelerometer data used to detect matter input from the x 600, y 602, and z 604 axis of the accelerometer, respectively. In at least one example, FIGS. 6A-C correspond to three different subjects ingesting twenty different boluses of an electrolyte solution with different volumes. For example, the subjects ingested twenty different boluses of an electrolyte solution with volumes varying from 0.5 to 4 ounces each. As shown, a high degree of repeatability from drink motion to drink motion is demonstrated. Other motions that could be confused with drinking were also performed.

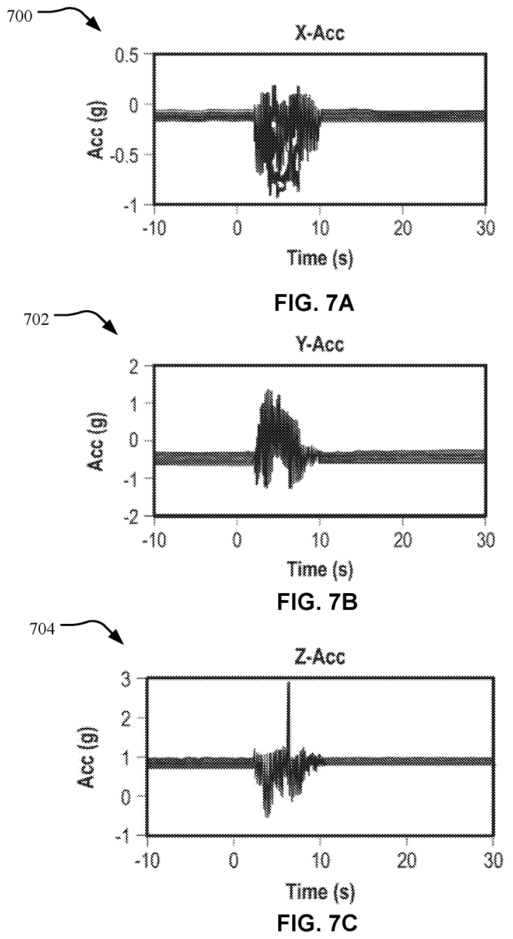

FIGS. 7A-C illustrate accelerometer data from three different subjects detecting actions similar but other than matter input from the x 700, y 702, and z 704 axis of the accelerometer, respectively. In at least one example, activities included picking up the phone, looking at the device, and touching ones hair were performed.

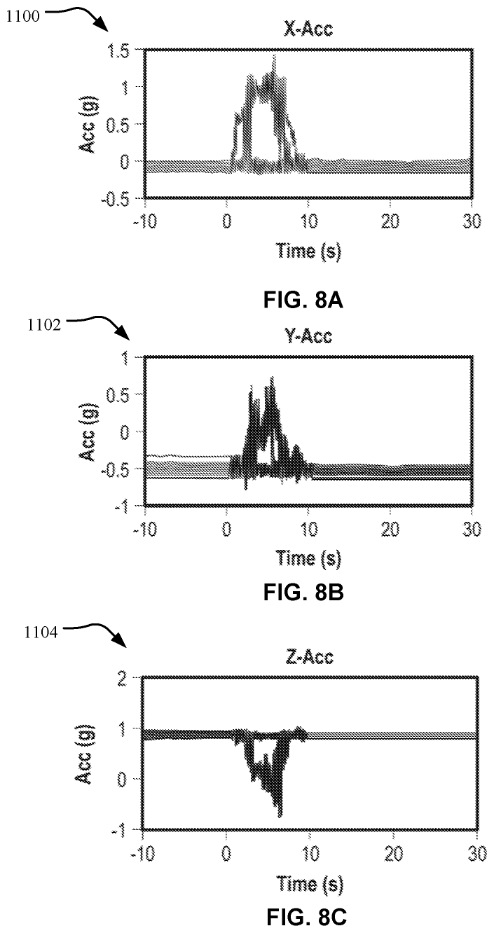

FIGS. 8A-C illustrate accelerometer data from three different subjects detecting actions which affect heart rate from the x 800, y 802, and z 804 axis of the accelerometer, respectively. In at least one example, subjects were asked to yawn.

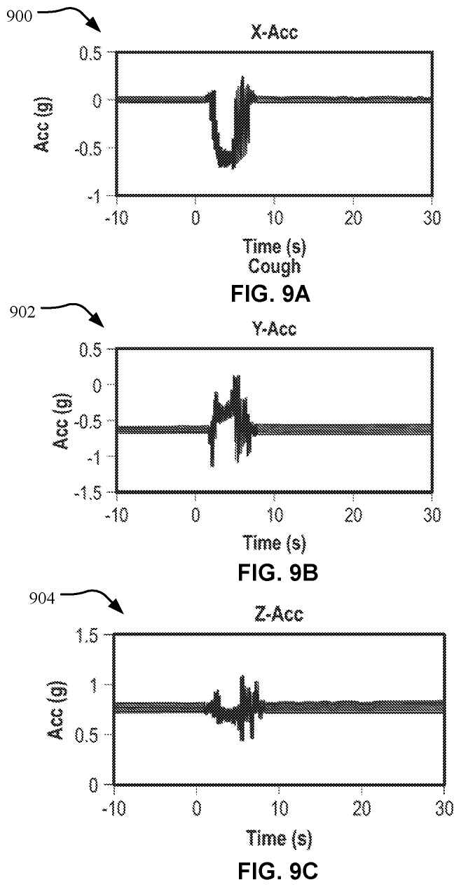

FIGS. 9A-C illustrate accelerometer data from three different subjects detecting a coughing action from the x 900, y 902, and z 904 axis of the accelerometer, respectively.

The data—including motion data and biological data—can be segmented and pre-processed. For example, pre-processing techniques can include, but are not limited to, dynamic time warping, dynamic tiling, and mathematical transformations such as Fourier transforms. Further, adaptive signal process methods can be used to adjust for different user 208s and wear variations. To distinguish between the drink and non-drink class of activities, algorithms can be used such as, for example, but not limited to, Normal Activity Recognition, Activity Thresholds, and k-nearest neighbors. For example, using a nearest-neighbor algorithm drinking events can be distinguished from non-drinking events with an accuracy better than 92%, a sensitivity better than 89% and a specificity better than 87%.

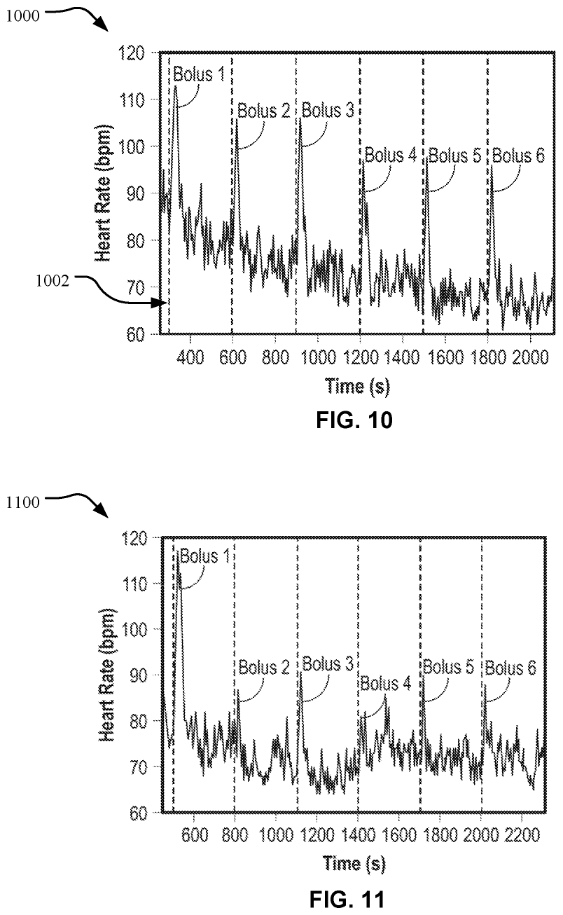

Another example study was also performed where heart rate was used to detect matter input, the first set of results 1000 are shown in FIG. 10. In this example, a subject ingested six boluses of a solution where the volume of the first bolus was 250 ml and the subsequent five boluses were each 153 ml. The dashed lines 1002 indicate the beginning of each drink event. As shown, the subject's heart rate surges shortly after each drink and last approximately the duration of the drinking event. The heart rate increases because one has to stop breathing while drinking or eating. As such, the supply of oxygen delivered is reduced and the heart increases the flow of blood to compensate for the oxygen deficit.

FIG. 11 shows a second example 1100 using heart rate to detect matter input wherein the same subject of FIG. 10, on a different day, ingested another six boluses of the same electrolyte solution wherein the first bolus was 250 ml and the subsequent five boluses were each 53 ml. As shown, the initial bolus produced a surge similar in amplitude and duration as example in FIG. 10. The subsequent boluses, smaller in volume, produced surges in heart rate that were significantly smaller in amplitude and duration than the subsequent boluses of FIG. 10.

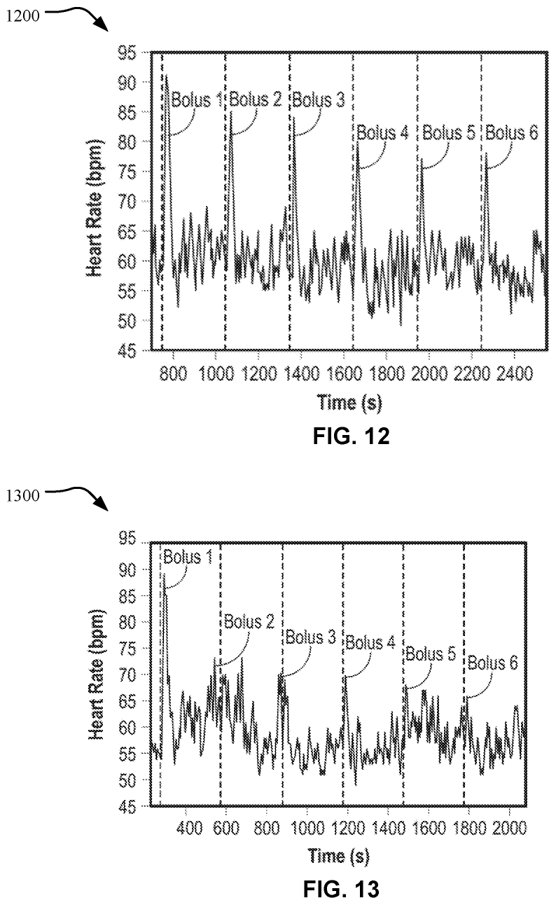

FIG. 12 shows a third example 1200 using heart rate to detect matter input. In at least one example, a different subject ingested six boluses similarly to the subject of FIGS. 10 and 11. The subject ingested six boluses of a solution where the volume of the first bolus was 250 ml and the subsequent five boluses were each 103 ml.

FIG. 13 shows a fourth example 1300 using heart rate to detect matter input wherein the same subject as FIG. 12 ingested boluses on a different day with the first bolus being 250 ml and the subsequent five boluses were each 27 ml.

FIGS. 10-13 illustrate that the amplitude and duration of the heart rate surge can be used to estimate the volume of ingested fluid. Also, the ability to estimate volume consumption from heart rate surges extends itself to multiple subjects. Furthermore, heart rate can also be used to detect output events, such as bowel movement, which also produces an increase in heart rate.

The increase in heart rate takes place because one has to stop breathing while drinking or eating. As such, the supply of oxygen delivered per liter of blood is reduced and the heart has to increase the flow of blood to keep core organs such as the brain, lungs, heart and liver, perfused, and it does so by increasing its pumping rate. Therefore, the surges are associated not only with drinking but with all matter ingestion events. In the case of smoking, for example, the increase in heart rate is associated with a decrease in oxygen available to the lungs during the smoke ingestion event. As such they can be used to detect input events and their integrated area can be used to estimate input volumes. Moreover, combining the heart rate signal with other signals, such as the accelerometer signal from the IMU, allows an algorithm to distinguish when the user of the device is drinking or eating. For example, in the study depicted in FIGS. 6A-C, it was observed that the combination of heart rate and IMU data resulted in a drink detection accuracy greater than 92%, a drink detection sensitivity greater than 90.5%, and a drink detection specificity greater than 86%.

Those skilled in the art also recognize that the Valsalva maneuver, associated with bowel movements, can also produce an increase in heart rate that can be detected by heart rate monitors and can thus be used to detect the output of matter. As in the case of heart rate surges during matter input, the onset of heart rate surges during the Valsalva maneuver indicates the beginning of the event while its integrated area increases proportionally to the volume and/or mass of matter output.

Mammalians (including humans) are capable of thermoregulation using, among other methods, sweat glands that induce a reduction of skin temperature by excreting sweat, composed mostly of water but also minerals, lactic acid and urea. Sweat excretion leads to evaporative cooling at the skin surface. As part of thermoregulation peripheral perfusion is also increased by vasodilation, leading to an increase in the volume of blood circulating in the periphery of skin. Evaporative cooling reduced the temperature of blood and its circulation leads to the decrease in temperature in core organs. Thermoregulation also leads to a reduction in hydration state of the user and this reduction (besides the loss of water due to sweat output) can be estimated by monitoring the difference between the skin temperature and ambient temperature: higher differences indicate higher sweat rates. This estimate can be improved taking into account perfusion information. For example, using skin total hemoglobin concentration (tHb) estimates provided by NIRS, as described in Publication WO 2016/191594, incorporated by reference herein.

Moreover, estimates of the user sweat rate can also be improved taking into account the amount of UV exposure, informing the algorithm that the user is exposed to direct sun light. Estimates can also be improved by taking into account the relative humidity of the surrounding air since the evaporative process is more effective when the air is dry. Estimates can also be improved by taking into account the difference in relative humidity of the air immediately in contact with skin and the surrounding air since evaporated sweat will increase the relative humidity of air immediately in contact with skin, and the differential relative humidity is a direct indication of the sweat rate. Moreover, the estimate can take into account the level of activity of the user, indicated by IMU data and/or by the heart rate, since the generation of heat is proportional to the level of activity of the user, and the larger the level of activity usually the higher is the sweat rate. Additionally, algorithms can take into account events scheduled in the users' calendars available in their smartphones. Calendar events including physical activities and workouts are also indicative of increased user activity, especially when combined with IMU and heart rate data to confirm their execution.

Moreover, the net balance of user water content can be adjusted by taking into account instantaneous and time-varying skin water content directly measured using NIRS, as described in U.S. Application Ser. No. 15/588,508. The adjustments in the net balance are then used to improve estimates of water input (in case of excess water) and output (in case of water deficit) events. Moreover, NIRS measurement of SmO₂ (muscle oxygenation) and/or HbO₂ (oxygenated hemoglobin concentration levels) and/or SpO₂ (arterial oxygenation saturation levels) provides information useful in estimating muscle and circulatory activity and, hence, caloric and water loss. That is, lower levels of SmO₂, HbO₂ and SpO₂ (significantly lower than baseline rest levels) are strong indicators of increased physical activity and, hence, increased caloric loss and increased water loss through sweat and perspiration.

During water absorption events, water is typically first ingested luminally before being absorbed by the digestive tract, at which point the water is transferred into the blood plasma. From the blood plasma, water is distributed throughout the body to arterioles and capillaries, where water becomes extracellular fluid before being osmotically absorbed by the cells in the body, thus becoming intracellular fluid. Cell membranes contain fatty tissue and are thus highly resistant to electric current while fluid is highly conductive. Thus, measuring the bioimpedance of the body provides one with an estimate of the ratio of intracellular over extracellular fluid content, thereby providing us with an estimate of fluid flow within the user body. Therefore, bioimpedance signals and its derivatives (galvanic skin response, skin resistance, skin conductance, electrodermal activity, psychogalvanic reflex, sympathetic skin response) are additional examples of biological signals that can be used in combination to estimate the occurrence and intensity of input and output events.

In addition, the mobile device or wearable device can use one or more of: an internal speaker, a microphone, or a camera to capture audio or video signals indicative of input or output events. Examples include the detection of sneezing, coughing, talking, crying or laughing, all of which increase the evaporative loss of water due to respiration. Moreover, crying and laughing indicate changes in mood—negative and positive, respectively. Moreover, audio signals can be used to improve the detection of liquid and/or solid output events.

Additionally, the mobile device or wearable device can directly or indirectly—for example, through an application—provide the user with information about input or output events. The information can include one or more of: the location of the event, the approximate time of the event, the duration of the event and estimated volume that was input and/or output. Additionally, the user can be asked to confirm, reject or edit the information. This information than can be stored in the device's long-term memory, and can be sent to a server in the cloud. Then, this information can be used by internal algorithms and/or by developers to improve the algorithm's ability to detect matter input or output events. The algorithms can also be improved, either by the action of programmers or through machine learning techniques, to personalize estimates of matter input/output status to the data produced by a specific user, thus improving estimates provided to that user over time. The data would also benefit developers over the long-run as more data becomes available to train their algorithms. Over time the improved algorithms could be deployed to the user base, assuring their long-term benefit. For example, user demographics enable developers to apply similar input/output and net balance estimation parameters to other users who share some or all of the demographics of previously recorded users.

Moreover, the location of the user or of a specific event can be determined by using a global positioning system present either in the wearable device or in the mobile device. Location can also be determined (precisely or approximately) using one of more wireless signals, allowing localization to be performed indoors, and within certain rooms of a certain building. Finally, wireless and global positioning data can be combined to improve accuracy and/or robustness of localization.

Examples of machine learning techniques that could be employed in at least one of the devices, the application, the remote computer, or the cloud computing system include neural networks, support vector machines, Bayesian learning, decision trees, reinforcement learning, linear regression models, or the like.

As previously discussed, the combination of data can be used to detect input and/or output events and can be used to distinguish between different types of events. For example, combining a heart rate signal with other signals, such as an accelerometer signal from an IMU, allows an algorithm to distinguish if the user is drinking or eating. This data can then be used to provide a user with information about matter input or output events. The user can be asked to confirm, reject, or edit the information, which can then be stored in the device's long-term memory. The information can then be used to improve the device's ability to detect matter input or output events by means of machine learning techniques, for example. Examples of machine learning techniques include, but are not limited to, neural networks, support vector machines, Bayesian learning, decision tress, reinforcement learning, linear regression models, or the like. Furthermore, algorithms can be used to provide suggestions on input amounts and duration to a user to maintain or increase performance. Numerous examples are provided herein to enhance understanding of the present disclosure. A specific set of statements are provided as follows.

Statement 1: A wearable device operable to provide recommendations to a user, the wearable device including at least one sensor operable to detect motion of the wearable device, a processor coupled to the at least one sensor, a biological sensor coupled to the processor and operable to detect a biological indicator of the user, and a memory that is operable to store instructions to cause the wearable device to: obtain at least one biological indicator of the user, correlate the biological indicator of the user with the detected motion in time, determine one or more input events or output events based on one or more of the at least one biological indicator or detected motion and determine a net balance of the user based on the determined one or more input events or output events.

Statement 2: The device of Statement 1, wherein each input event further includes an input activity and/or input duration.

Statement 3: The device of any one of the preceding Statements 1-2, wherein the memory is operable to store further instructions to create an input log for at least one of the determined input events.

Statement 4: The device of any one of the preceding Statements 1-3, wherein the input log includes an entry for each corresponding input event that includes at least one of the input activity, input duration, and/or input time.

Statement 5: The device of any one of the preceding Statements 1-4, wherein each output event further includes an output activity and/or output duration.

Statement 6: The device of any one of the preceding Statements 1-5, wherein the memory is operable to store further instructions to create an output log for at least one of the determined output events.

Statement 7: The device of any one of the preceding Statements 1-6, wherein the output log includes an entry for each corresponding output event that includes at least one of the output activity, output duration, and/or output time.

Statement 8: The device of any one of the preceding Statements 1-7, wherein the memory is operable to store further instructions to cause the wearable device to monitor the net balance and provide at least one notification that is associated with achieving one or more predefined goals.

Statement 9: The device of any one of the preceding Statements 1-8, wherein the one or more predefined goals being health or well-being.

Statement 10: The device of any one of the preceding Statements 1-9, further including a transmitter operable to transmit the detected biological indicator to at least one communication component of a mobile device.

Statement 11: The device of any one of the preceding Statements 1-10, wherein the memory is operable to store further instructions to cause the wearable device to display on a display a recommendation to the user for a next input event that can include at least one of an input activity, an input timing, and/or an input duration.

Statement 12: The device of any one of the preceding Statements 1-11, wherein the memory is operable to store further instructions to cause the wearable device to display on the display a recommendation to the user for a next output event that can include at least one of an output activity, an output timing, and/or an output duration.

Statement 13: The device of any one of the preceding Statements 1-12, wherein the biological sensor is operable to detect a heart rate, a heart rate variation, a respiration rate, a blood oxygen saturation level, skin temperature, skin perfusion, and/or sounds associated with a biological event.

Statement 14: The device of any one of the preceding Statements 1-13, wherein the memory is further operable to store instructions to cause the wearable device to display the determined input event on a display of the wearable device and receive confirmation and/or modification of the displayed input event.

Statement 15: The device of any one of the preceding Statements 1-14, further including at least one communication component operable to receive data from one or more external sensors or remote computer, wherein the remote computer is a cloud based computer system that includes one or more processors, one or more memories, and/or one or more storage devices.

Statement 16: The device of any one of the preceding Statements 1-15, wherein the at least one sensor operable to detect motion of the wearable device includes one or more of a gyroscope, an accelerometer, a magnetometer, and/or a global positioning system component.

Statement 17: The device of any one of the preceding Statements 1-16, further including an inertial motion unit operable to detect motions of a wrist of the user and the transmitter is operable to transmit the detected motions of the wrist to a mobile device.

Statement 18: The device of any one of the preceding Statements 1-17, wherein the memory is operable to store instructions to cause the mobile device to: obtain the data from the inertial motion unit with respect to the time, wherein the determination of the one or more input events is further based on the obtained data from the inertial motion unit.

Statement 19: The device of any one of the preceding Statements 1-18, further comprising determining a heart rate spike, wherein the time of the heart rate spike and motion detected from the inertial motion unit indicate a start of an input event.

Statement 20: The device of any one of the preceding Statements 1-19, wherein at least one predetermined motion indicates an end of the input event.

Statement 21: The device of any one of the preceding Statements 1-20, wherein one of the at least one predetermined motion is a return to a position prior to initiation of the input event.

Statement 22: The device of any one of the preceding Statements 1-21, further including one or more of a thermometer component operable to measure a temperature of skin of the user and surrounding ambient temperature, a near-infrared spectrometer operable to monitor chromophores that constitute a tissue of the user, a bioimpedance monitor, a photoplethysmograph monitor, a heart rate monitor, an ambient light sensor, an atmospheric pressure sensor, an altitude sensor, a relative humidity sensor, a scale, a microphone, and/or a localization sensor.

Statement 23: The device of any one of the preceding Statements 1-22, wherein the input event includes an input activity that includes intake of a solid, liquid, and/or gas.

Statement 24: The device of any one of the preceding Statements 1-23, wherein the input event includes an input activity that includes at least one of: eating, drinking, smoking, inhaling a mist or gas, temperature gain, muscle gain, bone mass gain, fat gain, calories gained, sleep gain, attention gain, alertness gain, and/or laughing.

Statement 25: The device of any one of the preceding Statements 1-24, wherein the output event includes an output activity that includes at least one of: perspiration, urination, defecation, excretion, temperature change, insensible fluid loss, fat loss, muscle loss, bone loss, evaporation, vomiting, sneezing, coughing, yelling, crying, and/or calories burnt.

Statement 26: The device of any one of the preceding Statements 1-25, wherein determining one or more input events is based on at least the at least one biological indicator and the detected motion; wherein determining one or more output events is based on at least the at least one biological indicator and/or detected motion.

Statement 27: A mobile device operable to determine habits of a user and make recommendations to a user, the mobile device including at least one sensor operable to detect at least one of motion of the mobile device or location of the device, a processor coupled to the one or more internal sensors, a display coupled to the processor and operable to display data received from the processor, and a memory coupled to the processor and operable to store instructions to cause the processor to: obtain at least one biological indicator of the user, correlate the at least one biological indicator of the user with the detected motion in time, determine one or more input events or output events based on one or more of the at least one biological indicator or detected motion and determine a net balance of the user based on the determined one or more events.

Statement 28: The device of Statement 27, wherein each input event further includes an input activity and/or input duration.

Statement 29: The device of any one of the preceding Statements 27-28, wherein the memory is operable to store further instructions to create an input log for at least one of the determined input events.

Statement 30: The device of any one of the preceding Statements 27-29, wherein the input log includes an entry for each corresponding input event that includes at least one of the input activity, input duration, and/or input time.

Statement 31: The device of any one of the preceding Statements 27-30, wherein each output event further includes an output activity and/or output duration.

Statement 32: The device of any one of the preceding Statements 27-31, wherein the memory is operable to store further instructions to create an output log for at least one the determined output events.

Statement 33: The device of any one of the preceding Statements 27-32, wherein the output log includes an entry for each corresponding output event that includes at least one of the output activity, output duration, and/or output time.

Statement 34: The device of any one of the preceding Statements 27-33, wherein the memory is operable to store further instructions to cause the wearable device to monitor the net balance and provide at least one notification that is associated with achieving one or more predefined goals.

Statement 35: The device of any one of the preceding Statements 27-34, wherein the one or more predefined goals being health or well-being.

Statement 36: The device of any one of the preceding Statements 27-35, wherein the memory is operable to store further instructions to cause the wearable device to display on a display a recommendation to the user for a next input event that can include at least one of an input activity, an input timing, and/or an input duration.

Statement 37: The device of any one of the preceding Statements 27-36, wherein the memory is operable to store further instructions to cause the wearable device to display on the display a recommendation to the user for a next output event that can include at least one of an output activity, an output timing, and/or an output duration.

Statement 38: The device of any one of the preceding Statements 27-37, wherein the biological sensor is operable to detect a heart rate, a heart rate variation, a respiration rate, a blood oxygen saturation level, skin temperature, skin perfusion, and/or sounds associated with a biological event.

Statement 39: The device of any one of the preceding Statements 27-38, wherein the memory is further operable to store instructions to cause the wearable device to display the determined input event on a display of the wearable device and receive confirmation or modification of the displayed input event.

Statement 40: The device of any one of the preceding Statements 27-39, further including at least one communication component operable to receive data from one or more external sensors or remote computer, wherein the remote computer is a cloud based computer system that includes one or more processors, one or more memories, and one or more storage devices.

Statement 41: The device of any one of the preceding Statements 27-40, wherein the at least one sensor operable to detect motion of the wearable device includes one or more of a gyroscope, an accelerometer, a magnetometer, or a global positioning system component.

Statement 42: The device of any one of the preceding Statements 27-41, further including an inertial motion unit is operable to detect motions of a wrist of the user and the transmitter is operable to transmit the detected motions of the wrist to a mobile device.

Statement 43: The device of any one of the preceding Statements 27-42, wherein the memory is operable to store instructions to cause the mobile device to: obtain the data from the inertial motion unit with respect to the time, wherein the determination of the one or more input events is further based on the obtained data from the inertial motion unit.

Statement 44: The device of any one of the preceding Statements 27-43, further comprising determining a heart rate spike, wherein the time of the heart rate spike and motion detected from the inertial motion unit indicate a start of an input event.

Statement 45: The device of any one of the preceding Statements 27-44, wherein at least one predetermined motion indicates an end of the input event.

Statement 46: The device of any one of the preceding Statements 27-45, wherein one of the at least one predetermined motion is a return to a position prior to initiation of the input event.

Statement 47: The device of any one of the preceding Statements 27-46, further including one or more of a thermometer component operable to measure a temperature of skin of the user and surrounding ambient temperature, a near-infrared spectrometer operable to monitor chromophores that constitute a tissue of the user, a bioimpedance monitor, a photoplethysmograph monitor, a heart rate monitor, an ambient light sensor, an atmospheric pressure sensor, an altitude sensor, a relative humidity sensor, a scale, a microphone, and/or a localization sensor.

Statement 48: The device of any one of the preceding Statements 27-47, wherein the input event includes an input activity that includes intake of a solid, liquid, and/or gas.

Statement 49: The device of any one of the preceding Statements 27-48, wherein the input event includes an input activity that includes at least one of: eating, drinking, smoking, inhaling a mist and/or gas, temperature gain, muscle gain, bone mass gain, fat gain, calories gained, sleep gain, attention gain, alertness gain, and/or laughing.

Statement 50: The device of any one of the preceding Statements 27-49, wherein the output event includes an output activity that includes at least one of: perspiration, urination, defecation, excretion, temperature change, insensible fluid loss, fat loss, muscle loss, bone loss, evaporation, vomiting, sneezing, coughing, yelling, crying, and/or calories burnt.

Statement 51: The device of any one of the preceding Statements 27-50, wherein determining one or more input events is based on at least the at least one biological indicator and the detected motion; wherein determining one or more output events is based on at least the at least one biological indicator and detected motion.

Statement 52: A mobile device system operable to provide recommendations to a user including a device of Statement 1 and any combination of Statements 2-26 or the device of Statement 27 and any combination of Statements 28-51 operable to receive data from the device of Statement 1 and any combination of Statements 2-26 or the device of Statement 27 and any combination of Statements 28-51 or a remote computer.

Statement 53: The system of Statement 52, further including the remote computer comprising a processor and a memory that is operable to store instructions to: obtain at least one biological indicator of the user, correlate the biological indicator of the user with the detected motion in time, determine one or more input or output events based on one or more of the at least one biological indicator or detected motion, each event including an activity and a duration, and determine a net balance of the user based on the determined one or more events.

Statement 54: The system of any of the preceding Statements 52-53, wherein the remote computer is a cloud based computer system that includes one or more processors, one or more memories, and/or one or more storage devices.

Statement 55: The system of any of the preceding Statements 52-54, wherein each input event further includes an input activity and/or input duration.

Statement 56: The system of any of the preceding Statements 52-55, wherein the memory is operable to store further instructions to create an input log for at least one of the determined input events.

Statement 57: The system of any of the preceding Statements 52-56, wherein the input log includes an entry for each corresponding input event that includes at least one of the input activity, input duration, and/or input time.

Statement 58: The system of any of the preceding Statements 52-57, wherein each output event further includes an output activity and output duration.

Statement 59: The system of any of the preceding Statements 52-58, wherein the memory is operable to store further instructions to create an output log for at least one of the determined output events.

Statement 60: The system of any of the preceding Statements 52-59, wherein the output log includes an entry for each corresponding output event that includes at least one of the output activity, output duration, and/or output time.

Statement 61: The system of any of the preceding Statements 52-60, wherein the memory is operable to store further instructions to cause the wearable device to monitor the net balance and provide at least one notification that is associated with achieving one or more predefined goals.

Statement 62: The system of any of the preceding Statements 52-61, wherein the one or more predefined goals being health or well-being.

Statement 63: The system of any of the preceding Statements 52-62, further including a transmitter operable to transmit the detected biological indicator to at least one communication component of the device of Statement 1 and any combination of Statements 2-26 or the device of Statement 27 and any combination of Statements 28-51.

Statement 64: The system of any of the preceding Statements 52-63, wherein the memory is operable to store further instructions to cause the device of Statement 1 and any combination of Statements 2-26 or the device of Statement 27 and any combination of Statements 28-51 to display on a display a recommendation to the user for a next input event that can include at least one of an input activity, an input timing, and/or an input duration.

Statement 65: The system of any of the preceding Statements 52-64, wherein the memory is operable to store further instructions to cause the device of Statement 1 and any combination of Statements 2-26 or the device of Statement 27 and any combination of Statements 28-51 to display on the display a recommendation to the user for a next output event that can include at least one of an output activity, an output timing, and/or an output duration.

Statement 66: The system of any of the preceding Statements 52-65, wherein the memory is further operable to store instructions to cause the device of Statement 1 and any combination of Statements 2-26 or the device of Statement 27 and any combination of Statements 28-51 to display the determined input event on a display of the device of Statement 1 and any combination of Statements 2-26 or the device of Statement 27 and any combination of Statements 28-51 and receive confirmation or modification of the displayed input event.

Statement 67: The system of any of the preceding Statements 52-66, wherein the memory is operable to store instructions to cause the mobile device to: obtain the data from the inertial motion unit of the device of Statement 1 and any combination of Statements 2-26 or the device of Statement 27 and any combination of Statements 28-51 with respect to the time, wherein the determination of the one or more input events is further based on the obtained data from the inertial motion unit.

Statement 68: The system of any of the preceding Statements 52-67, further comprising determining a heart rate spike, wherein the time of the heart rate spike and motion detected from the inertial motion unit indicate a start of an input event.

Statement 69: The system of any of the preceding Statements 52-68, wherein at least one predetermined motion indicates an end of the input event.

Statement 70: The system of any of the preceding Statements 52-69, wherein one of the at least one predetermined motion is a return to a position prior to initiation of the input event.

Statement 71: The system of any of the preceding Statements 52-70, wherein the input event includes an input activity that includes intake of a solid, liquid, and/or gas.

Statement 72: The system of any of the preceding Statements 52-71, wherein the input event includes an input activity that includes at least one of: eating, drinking, smoking, inhaling a mist and/or gas, temperature gain, muscle gain, bone mass gain, fat gain, calories gained, sleep gain, attention gain, alertness gain, and/or laughing.

Statement 73: The system of any of the preceding Statements 52-72, wherein the output event includes an output activity that includes at least one of: perspiration, urination, defecation, excretion, temperature change, insensible fluid loss, fat loss, muscle loss, bone loss, evaporation, vomiting, sneezing, coughing, yelling, crying, and/or calories burnt.

Statement 74: The system of any of the preceding Statements 52-73, wherein determining one or more input events is based on at least the at least one biological indicator and the detected motion; wherein determining one or more output events is based on at least the at least one biological indicator and detected motion.

Statement 75: The device of any of the preceding Statements 1-8, wherein the one or more predefined goals being maintaining a predefined hydration level.

The description above includes example systems, methods, techniques, instruction sequences, and/or computer program products that embody techniques of the present disclosure. However, it is understood that the described disclosure can be practiced without these specific details.

It is believed that the present disclosure and many of its attendant advantages will be understood by the foregoing description, and it will be apparent that various changes can be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various examples, it will be understood that these examples are illustrative and that the scope of the disclosure is not limited to them. Many variations, modifications, additions, and improvements are possible. More generally, examples in accordance with the present disclosure have been described in the context of particular implementations. Functionality can be separated or combined in blocks differently in various examples of the disclosure or described with different terminology. These and other variations, modifications, additions, and improvements can fall within the scope of the disclosure as defined in the claims that follow.

1. A wearable device operable to provide recommendations for a user, the wearable device comprising:
at least one sensor operable to detect motion of the wearable device;
a processor coupled to the at least one sensor;
a biological sensor coupled to the processor and operable to detect a biological indicator of the user; and
a memory that is operable to store instructions to cause the wearable device to:
obtain at least one biological indicator of the user;
correlate the biological indicator of the user with the detected motion in time;
determine one or more input events based on one or more of the at least one biological indicator or detected motion, each input event including an input activity and input duration;
create an input log for every determined one or more input events, wherein the input log includes an entry for each corresponding input event that includes at least one of the input activity, input duration, and/or input time;
determine one or more output events based on one or more of the at least one biological indicator or detected motion, each output event including an output activity and/or output duration;
create an output log for every determined one or more output events, wherein the output log includes an entry for each corresponding output event that includes at least one of the output activity, output duration, and/or output time; and
determine a net balance of the user based on the determined one or more input events and one or more output events, wherein the net balance is determined by the one or more input events minus the one or more output events.

2. The wearable device of claim 1, wherein the memory is operable to store further instructions to cause the wearable device to monitor the net balance and provide at least one notification that is associated with achieving one or more predefined goals.

3. The wearable device of claim 2, wherein the one or more predefined goals being maintaining a predefined hydration level, health, and/or well-being.

4. (canceled)

5. The wearable device of claim 1, further comprising a transmitter operable to transmit the detected biological indicator to at least one communication component of a mobile device.

6. The wearable device as recited in claim 5, further comprising an inertial motion unit operable to detect motions of a wrist of the user and the transmitter is operable to transmit the detected motions of the wrist to a mobile device.

7. The wearable device as recited in claim 6, wherein the memory is operable to store instructions to cause the mobile device to:
obtain the data from the inertial motion unit with respect to the time, wherein the determination of the one or more input events is further based on the obtained data from the inertial motion unit.

8. The wearable device as recited in claim 7, wherein the time of a heart rate spike and motion detected from the inertial motion unit indicate a start of an input event.

9. The wearable device as recited in claim 8, wherein at least one predetermined motion indicates an end of the input event.

10. The wearable device as recited in claim 9, wherein one of the at least one predetermined motion is a return to a position prior to initiation of the input event.

11. The wearable device of claim 1, wherein the memory is operable to store further instructions to cause the wearable device to display on a display a recommendation to the user for a next input event that includes an input activity, an input timing, and/or an input duration.

12. (canceled)

13. The wearable device of claim 1, wherein the memory is operable to store further instructions to cause the wearable device to display on the display a recommendation to the user for a next output event that includes an output activity, an output timing, and/or an output duration.

14. The wearable device of claim 1, wherein the biological sensor is operable to detect a heart rate, a heart rate variation, a respiration rate, a blood oxygen saturation level, skin temperature, skin perfusion, and/or sounds associated with a biological event.

15. The wearable device as recited in claim 1, wherein the memory is further operable to store instructions to cause the wearable device to display the determined input event on a display of the wearable device and receive confirmation or modification of the displayed input event.

16. The wearable device as recited in claim 1, further comprising at least one communication component operable to receive data from one or more external sensors or remote computer, wherein the remote computer is a cloud based computer system that includes one or more processors, one or more memories, and one or more storage devices.

17. The wearable device as recited in claim 1, wherein the at least one sensor operable to detect motion of the wearable device includes one or more of a gyroscope, an accelerometer, a magnetometer, or a global positioning system component.

18. The wearable device as recited in claim 1, further comprising one or more of a thermometer component operable to measure a temperature of skin of the user and surrounding ambient temperature, a near-infrared spectrometer operable to monitor chromophores that constitute a tissue of the user, a bioimpedance monitor, a photoplethysmograph monitor, a heart rate monitor, an ambient light sensor, an atmospheric pressure sensor, an altitude sensor, a relative humidity sensor, a scale, a microphone, and/or a localization sensor.

19. The wearable device as recited in claim 1, wherein the input activity includes intake of a solid, liquid, and/or gas.

20. The wearable device as recited in claim 19, wherein the input activity includes at least one of: eating, drinking, smoking, inhaling a mist and/or gas, temperature gain, muscle gain, bone mass gain, fat gain, calories gained, sleep gain, attention gain, alertness gain, and/or laughing.

21. The wearable device as recited in claim 1, wherein the output activity includes at least one of: perspiration, urination, defecation, excretion, temperature change, insensible fluid loss, fat loss, muscle loss, bone loss, evaporation, vomiting, sneezing, coughing, yelling, crying, or calories burnt.

22. The wearable device as recited in claim 1, wherein determining one or more input events is based on at least the at least one biological indicator and the detected motion; wherein determining one or more output events is based on at least the at least one biological indicator and detected motion.

23.-28. (canceled)
